(19) United States
(12) Patent Application Publication
(10) Pub. No.: US 2024/0075228 A1
(43) Pub. Date: Mar. 7, 2024

(54) Intelligent Laryngeal Mask Airway Device with Visualization

(71) Applicant: IntraVu, Inc., Redwood City, CA (US)

(72) Inventor: Stephanos Papademetriou, Woodside, CA (US)

(21) Appl. No.: 17/990,574

(22) Filed: Nov. 18, 2022

Related U.S. Application Data
(60) Provisional application No. 63/281,490, filed on Nov. 19, 2021.

(51) Int. Cl. A61M 16/04

(52) U.S. Cl. CPC A61M 16/0447; A61M 16/0486; A61M 16/0488; A61M 2016/0027

(57) ABSTRACT

A laryngeal mask airway device that establishes pulmonary breathing of a patient and receives a endotracheal tube includes an airway tube with a distal end mask, the mask having a peripheral cuff to form a seal around the patient's laryngeal inlet. An endoscope channel is formed separately from the airway tube so as to receive an endoscope capable of illuminating and viewing the laryngeal anatomy. The endoscope channel may be closed by a transparent viewing window at a distal end. In an embodiment the endoscope channel has a mechanical asymmetry to ensure proper insertion of the visualizaton stylet. The airway device may include an additional channel for monitoring pressure in the laryngeal region. A visualization stylet to be used with the airway device is preferably connected wirelessly to display equipment. The airway device can also include markings to be imaged by the visualization stylet during insertion, for correct placement and to assess dimensions of a patient's anatomical features.

### BACKGROUND OF THE INVENTION

This application claims benefit of provisional application Ser. No. 63/281,490, filed Nov. 19, 2021.

The present invention relates to a laryngeal mask airway (LMA) device with an additional visualization channel. More specifically, the present invention relates to an intubating laryngeal mask airway (ILMA) device with a secondary visualization channel.

The LMA is a well-known device that is useful for establishing airways in unconscious patients. U.S. Pat. No. 4,509,514 is one of the many publications that describe LMA devices. Such devices have been in use for many years and offer an alternative to the older, even better known, endotracheal tube (ETT). For close to one hundred years, endotracheal tubes comprising a long slender tube with an inflatable balloon located at the tube's distal end have been used for establishing airways in unconscious patients. In operation, the endotracheal tube's distal end is inserted through the mouth of the patient, past the patient's laryngeal inlet (or glottic opening), and into the patient's trachea. Once so positioned, the balloon is inflated in order to form a seal with the interior lining of the trachea. After this seal is established, positive pressure may be applied to the tube's proximal end to ventilate the patient's lungs. Also, the seal between the balloon and the inner lining of the trachea protects the lungs from aspiration (e.g., the seal prevents material regurgitated from the stomach from being aspirated into the patient's lungs).

Although they have been enormously successful, ETTs suffer from several major disadvantages:
(1) The principal disadvantage of the endotracheal tube relates to the difficulty of properly inserting the tube. Inserting an ETT into a patient is a procedure that requires a high degree of skill. Even for skilled practitioners, insertion of an endotracheal tube is sometimes difficult or not possible. In many instances, the difficulty of inserting an ETT can tragically lead to the death of a patient because it was not possible to establish an airway in the patient with sufficient speed.
(2) Inserting an ETT normally requires manipulations of the patient's head and neck. These necessary manipulations make it difficult, or undesirable, to insert an endotracheal tube into a patient who may be suffering from a neck injury.
(3) Inserting an ETT further requires the patient's jaw to be forcibly opened widely and tongue moved out of the way. Such manipulations require the use of a laryngoscope, or video laryngoscope. In order to perform intubation successfully with direct laryngoscopy, one needs to create a line of sight for the operator to visualize the vocal cords directly. This necessitates placing the patient in an appropriate position before direct laryngoscopy to anteriorly displace the mandible, tongue, and other soft tissue, which can then clear the path for subsequent ETT entry. This potentially stressful maneuver may result in oral and dental trauma.

In patients with limitations in cervical mobility or mouth opening, the line of sight may not be achievable, resulting in poor laryngeal views, and difficult or impossible intubation (Thong SY, Lim Y (2009) “Video and optic laryngoscopy assisted tracheal intubation-the new era.” Anaesth Intensive Care 37:219-233)

In contrast to the ETT, it is relatively easy to insert a LMA into a patient and thereby establish an airway to ventilate a patient.
a. The LMA is considered a “forgiving” device in that even if it is inserted improperly, it still tends to establish an airway. Accordingly, the LMA is often thought of as a “life saving” device.
b. Also, the LMA may be inserted with only relatively minor manipulations of the patient's head, neck, and jaw. Thus, for example, patients with prior cervical spine surgery may not be able to intubate easily.
c. Further, the LMA provides for ventilation of the patient's lungs without requiring contact with the sensitive inner lining of the trachea and the size of the airway established is typically significantly larger than the size of the airway established with an endotracheal tube.

Largely due to these advantages, the LMA has enjoyed increasing popularity in recent years.

U.S. Pat. Nos. 5,303,697 and 6,079,409 describe examples of a type of prior art device that may be referred to as an “intubating laryngeal mask airway device (ILMA).” The ILMA is useful for facilitating insertion of an endotracheal tube (ETT). After an ILMA device has been located in the patient, the device can act as a guide for a subsequently inserted endotracheal tube. Use of the laryngeal mask airway device in this fashion facilitates what is commonly known as “blind insertion” of the endotracheal tube. Only minor movements of the patient's head, neck, and jaw are required to insert the intubating laryngeal mask airway device, and once the device has been located in the patient, the endotracheal tube may be inserted with virtually no additional movements of the patient. This stands in contrast to the relatively large motions of the patient's head, neck, and jaw that would be required if the endotracheal tube were inserted without the assistance of the intubating laryngeal mask airway device.

Although these constructs offer an alternative to intubation with the aid of a laryngoscope, the passage of the ETT is performed blindly, based solely on the fact that the LMA's airway output port is most likely in front of the trachea. Thus, intubating with an ETT can prove less effective especially in difficult to intubate patients.

Several prior art designs then took the next step by adding visualization as means of aiding the insertion of an LMA or ETT.

U.S. Pat. No. 5,682,880 describes an improved design of an ILMA that contains a stiff reinforced member that is removable to assist in the placement of the LMA. The channels used for the insertable reinforced member can after placement be used by a removable fiberoptic imaging scope for visualization after insertion of the LMA and better aid the insertion of an ETT. Furthermore, since the visualization channel is open at the distal end, the fiberscope must be reprocessed or sterilized which adds complications, delays and cost. U.S. Pat. No. 6,929,600 describes a different method of visualizing the ETT insertion into the trachea by including a removable visualization stylet that runs through the ETT. This way the distal end of the ETT can be monitored and guided during insertion under direct visualization. Although a convenient way to add visualization, such system will still require the aid of a laryngoscope for inserting such ETT and thus inherits all the negative aspects described earlier of utilizing a laryngoscope for intubating a patient. Also, the ETT must be disposable, or reprocessed.

U.S. Pat. No. 7,128,071 describes an ILMA with embedded visualization of fiberoptic imaging bundles. It has been modified with the addition of built-in fiberoptic imaging that can provide a view of the larynx and observation of the endotracheal tube passing through the LMA and into the vocal cords. But imaging through fiberoptics is poor, and the fact that the imaging fiberoptic bundles are embedded in the LMA increase the cost of it. It functions similarly to the blind ILMAs of the prior art, but the design calls for a stiff element and rigid handle in the ILMA as well as an epiglotic cover to prevent blocking of the airway distal channel. Japanese patent publication 2008528131A describes a similar ILMA but with embedded videoscope instead of fiberoptic imaging.

Additionally, all ILMA prior art does not specifically describe the possible removal of the LMA once the ETT is inserted in the trachea and secured in place by inflating its distal balloon. Commercial products offer awkward extension elements that the user needs to place at the proximal end of the ETT (thus stop the patient's intubation) in order to be able to hold onto something while removing the LMA by sliding it off the ETT (see for example YouTube video youtube/w5HNhG93lj4?t=172).

### SUMMARY OF THE INVENTION

It is the object of this invention to describe a novel intelligent design of an LMA that includes a way for an insertable visualization stylet to monitor its insertion into the mouth as well as monitor the stability and securement of its final resting place during ventilation and alert the anesthesiologist when it is moved out of place. This not only can provide powerful validation of the placement of the LMA, but also added safety of the securement of its final placement by continually monitoring it.

According to another aspect and embodiment of the present invention, the visualization LMA can act as a laryngoscope to assist in the accurate placement of an ETT (thus become an Intubation LMA or ILMA) without any of the downsides associated with using direct laryngoscopy for placement of an ETT but with all the benefits and the ease of use of an LMA especially in critical or difficult to intubate situations. In this aspect of the invention the LMA is designed so that it can easily be pealed off of the ETT (without having to be pulled off of it by running the whole LMA along the length of the ETT) thus allowing for easier and faster removal of the LMA after the ETT tube is placed into the trachea and its distal balloon is inflated to secure the ETT in place.

There is yet another object of this invention, where once the view of the patient's airway is digitized and communicated to the user through a computer, smart phone, digital pad, or monitor, data can be collected about the individual patient and stored in the cloud for further analysis. Such large data sets can help develop algorithms for better assisting the physician with selecting properly sized hardware in future surgeries. Additionally, such intelligent ventilation/intubation system can prove to be a great training tool, where a senior anesthesiologists could be monitoring multiple intubations performed by physicians or nurses under training at the same time and offering assistance even in real time.

Finally it is the object of this invention to further impart intelligence to the LMA by continually monitoring its placement through visualization as well as monitoring the pressure inside the cuff pocket of the mask of the LMA.

### DESCRIPTION OF THE DRAWINGS

FIG. 1 is a perspective view showing two embodiments of an LMA in accordance with the invention.

FIG. 2 includes two views indicating the LMA placed within a patient.

FIG. 3 is a schematic view indicating the LMA placed correctly within a patient and a visualization stylet being inserted into the LMA.

FIG. 4 includes FIGS. 4A through 4D and shows different embodiments of a visualization stylet distal tip and distal clear window.

FIG. 5 includes two views indicating different embodiments for monitoring live video from the visualization stylet of the invention.

FIG. 6A is a schematic view indicating monitoring of a plurality of intubation cases by a supervisor, with signals from all LMA placements sent to the supervisor.

FIG. 6B is a perspective view and a detail showing the procedure of insertion of a visualization stylet through a visualization channel of the LMA.

FIG. 7 includes three detail views indicating use of software for re-orienting an image that might be rotated during installation or use of the LMA.

FIG. 8 includes two perspective views of the LMA, with three channels in the LMA and including an inflatable cuff and a non-inflatable cuff.

FIG. 9 includes two views and indicates the LMA with inflatable cup and ETT inserted through the LMA for intubation.

FIG. 10 includes FIGS. 10A through 10E and indicates an aspect of the invention in which the LMA can be peeled off the ETT as the LMA is removed after ETT placement.

FIG. 11 is a cross section view detailing particular embodiments of a peelable LMA.

FIG. 12, a schematic including two views, shows a movable pressure membrane at the distal end of a pressure monitoring channel of the LMA, for detecting changes in pressure below the LMA that can indicate movement of the cuff or other problems.

### DESCRIPTION OF PREFERRED EMBODIMENTS

Design of an LMA with Visualization Channel:

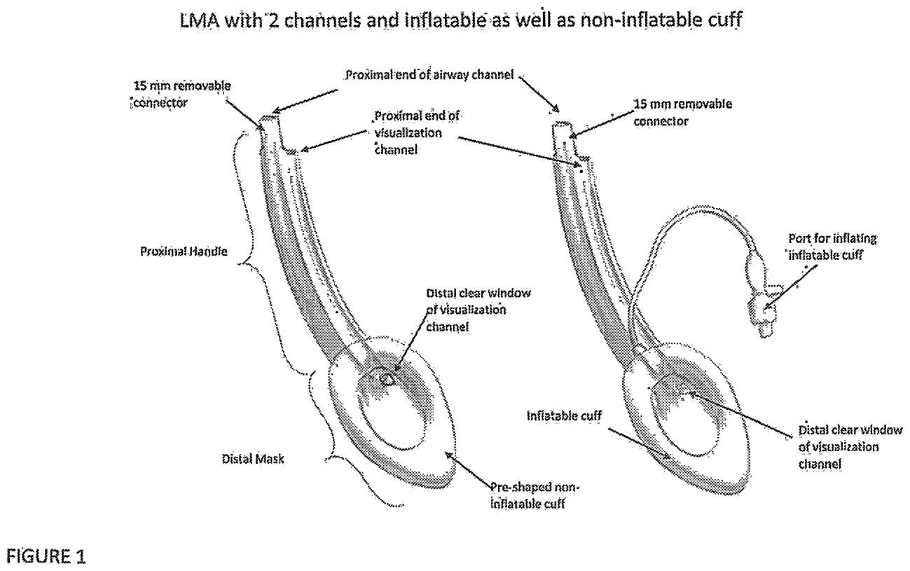

The LMA of this invention has two components: a proximal softer handle that attaches to a second distal piece that is shaped like the typical mask of an LMA. The distal mask portion of the LMA can have an inflatable cuff around its perimeter or a non-inflatable cuff that provides rather an anatomically pre-shaped cuffless seal made out of a soft cushioning material. Such non-inflatable cuff can be made of a hydrophilic silicon that is atraumatic to aid in a blind insertion of the LMA through the mouth. The distal tip of the cuff is optimized to block and seal the gastric path and to functionally separate the digestive and respiratory tracts. See FIG. 1, showing the LMA with only two channels and an inflatable and non-inflatable cuff.

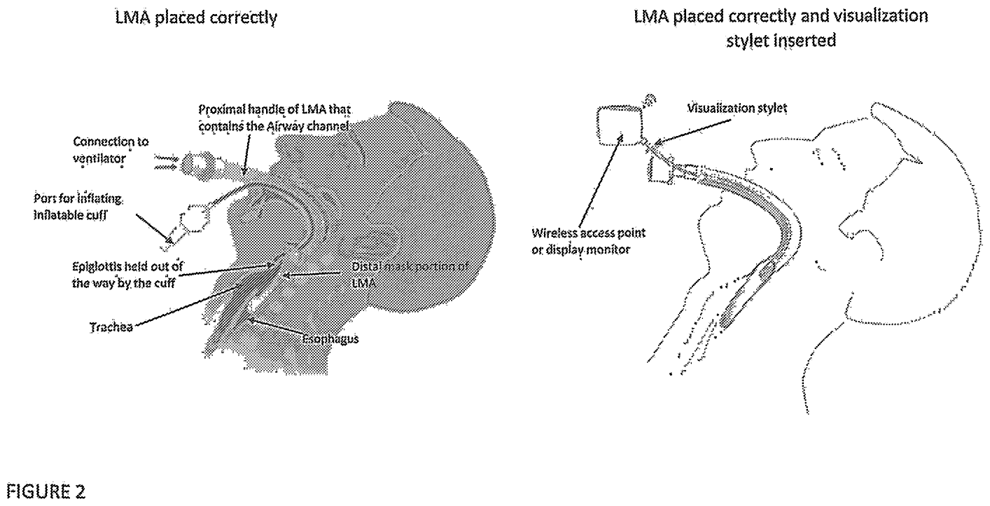

When the LMA is pushed into the patient's mouth the softer handle conforms to the shape of the patient's anatomy while pushing the cuffed distal mask portion into the patient's larynx/pharynx to make a seal around the proximal end of the trachea and allow for ventilation (see FIG. 2).

Along the length of the proximal handle there are at least two channels running along its length from the proximal end all the way to the distal end of the handle and are communicating with similar openings on the distal mask end of the LMA. This way the channels of the LMA communicate from the proximal end of the handle all the way to the distal end face of the distal mask portion of the LMA. The main channel allows for air to be delivered for ventilation (airway channel). The air way channel is outfitted with a standard 15 mm connector that can be removable. Once the LMA is pushed and secured in its final resting place, ventilation can commence.

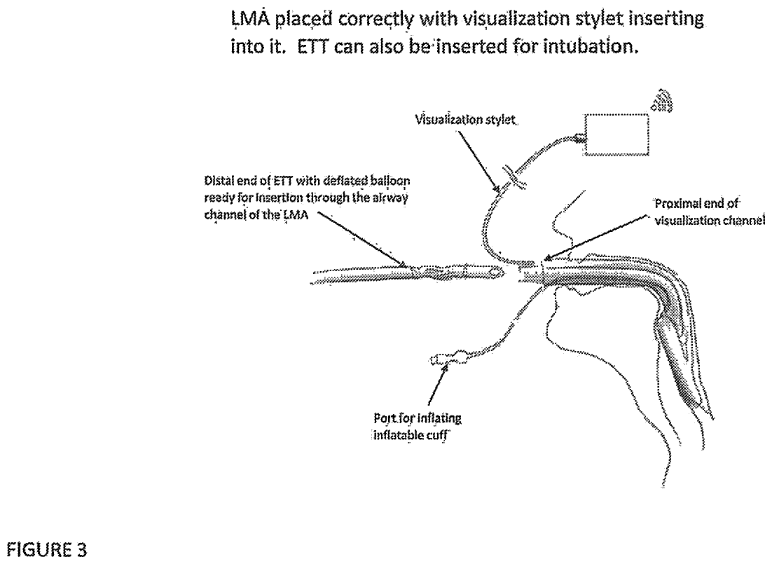

A second channel runs next to the main airway channel that can allow the insertion of a removable visualization stylet to be pushed through it. Such stylet is pushed all the way to the distal end of the visualization channel to monitor the distal end of the LMA as it is inserted into the patient's mouth, as well as monitor whether the LMA has moved and whether it needs to be re-positioned (see FIG. 3; stylet getting inserted into the side visualization channel).

The location and angle of the distal end of the visualization channel in the mask portion of the LMA is such that when the visualization stylet is inserted and pushed down this channel its field of view will be centered around the vocal cords of the patient (proximal end of the trachea). Even if it is not perfectly centered, modern videoscopes can be designed with large field of view (120 deg or more) that will ensure that the anatomies of interest will always be in view to ensure proper placement of the LMA and successful ventilation.

The distal end of the visualization channel is preferably closed and consists of a clear window material so that light and images can be transmitted without significant additional loss or aberrations. This clear window at the end of the visualization channel can act as a sterile barrier. Such design will ensure that the visualization stylet needs no reprocessing or sterilization to be re-used since it never comes in contact with the patient. The outside surface of the window may also be coated with a hydrophobic coating to prevent mucosa or saliva from sticking to it. Additionally, the clear window at the distal end of the LMA must be positioned in a location along the surface of the mask that also is less likely to get covered by mucosa or saliva.

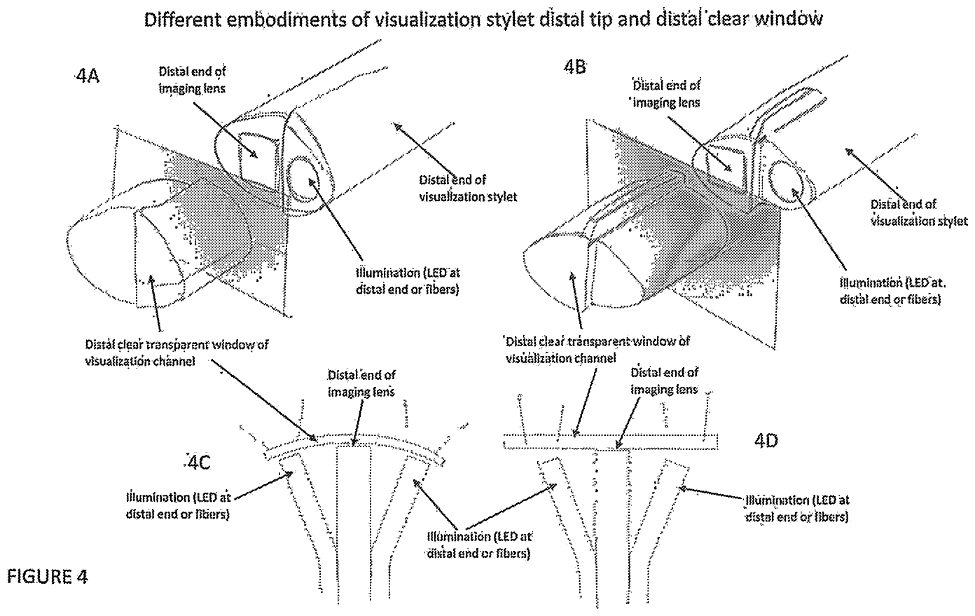

Since the visualization stylet contains both the imaging sensor and illumination at its distal end, reflections of the illumination light off the surfaces of the distal clear window may travel back into the optical system of the camera. Such reflections could completely ruin the contrast of the image. Thus, it is a preferred embodiment of this invention that the distal clear window and the stylet be designed so that such reflections cannot make it back into the imaging sensor. (FIG. 4; visualization end window designs). One can design the tip of the visualization stylet so the illumination source is slightly in front of the imaging sensor. The clear window on the LMA base of the mask then has the same accommodation in its mechanical design (FIG. 4A). In such clear window design, reflections of the illumination light off the window can be prevented from ever getting to the imaging lens. The length of stylet that the illumination portion of the stylet extends beyond the sensor must be designed as to prevent reflections off the clear window from making it to the sensor, while at the same time the small obstruction it creates in the view is clinically insignificant. In FIG. 4B the sensor is ahead of the illumination source. In this embodiment, the sensor will block some of the light from the illumination source, but the view will be unobstructed. But if it is designed just properly, the amount of light blocked is clinically insignificant. There are other ways of accomplishing the same (among many) as indicated in FIGS. 4C and 4D where the illumination source is angled away from the imaging sensor so that reflections off the clear window never make it to the sensor. In such embodiments the distal end can also be in physical contact with the window. An optically clear and index matching liquid can also be used to eliminate the first Fresnel reflection off of the inside surface of the clear window. The second Fresnel reflection off the distal end of the window can be designed not to make it back to the imaging sensor.

The visualization stylet should be made of a malleable or flexible material so that it can conform to the shape of the visualization channel as it is inserted through it. It can be a fiberscope or preferably a videoscope that is communicating with image processing hardware and illumination source proximal to it. The light source of the stylet can also be light emitting diodes (LEDs) positioned at the distal tip of the stylet around the digital imaging sensor. Such hardware can then provide live video of the field of view of the distal end of the stylet (via wire or wirelessly) to a display, or tablet, or smart phone. In other words, the proximal end of the stylet can connect directly into a tablet for image processing and display of video on its integrated display, or to an access point which can transmit video wirelessly to a tablet, smart phone, or external display correspondingly equipped to receive such signal from the access point.

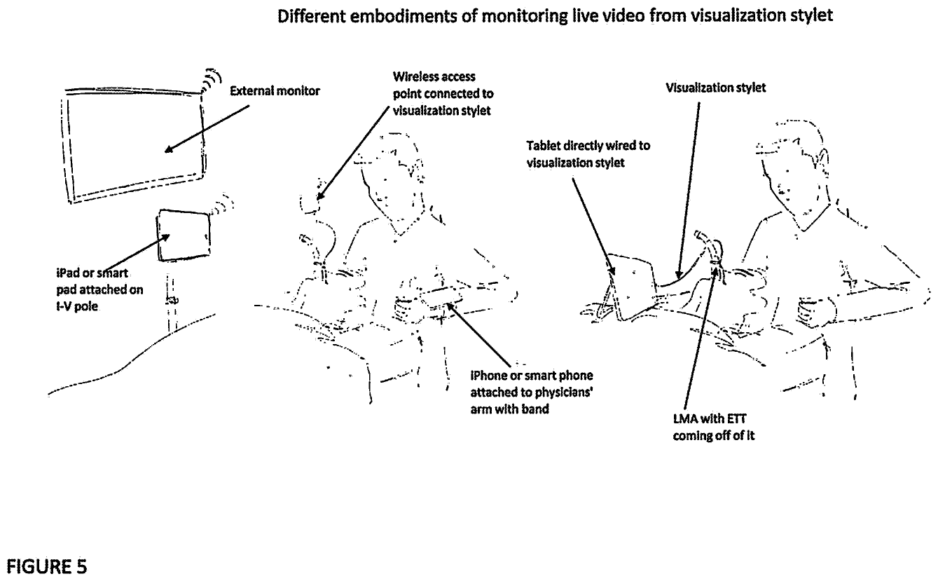

When wirelessly connected to an external tablet or smart phone (receiver device), a corresponding app must be installed to such receiver device in order to communicate with the access point and display video on its screen. The wireless communication between the access point and receiver can be encrypted to comply with HIPPA rules. Data can be stored onto the receiver device or can be transferred to a PACS compatible server for storage, evaluation along with the patient's other electronic medical records (see FIG. 5; different connections of the stylet—into tablet, into wireless transmitter and then to smart phone or iPad on an I-V pole).

Remote Monitoring and Training:

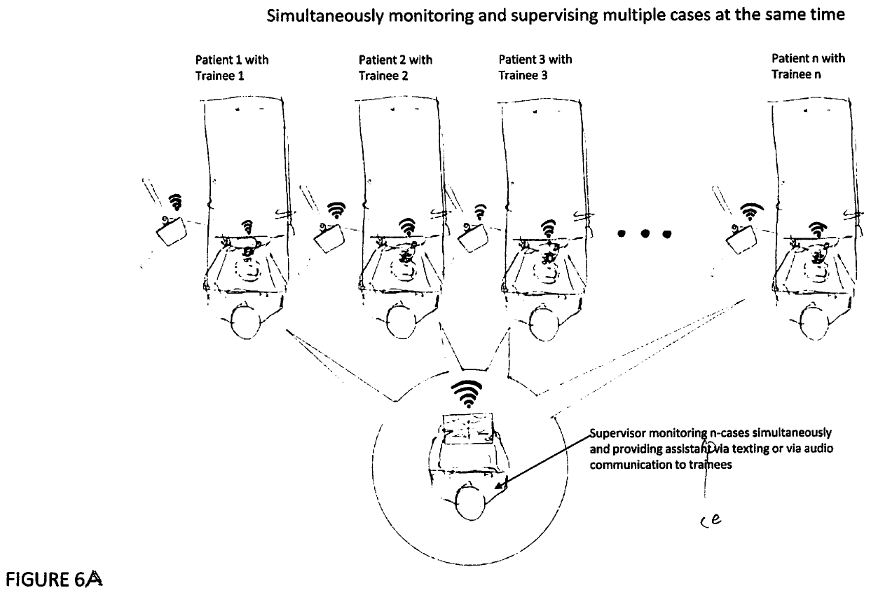

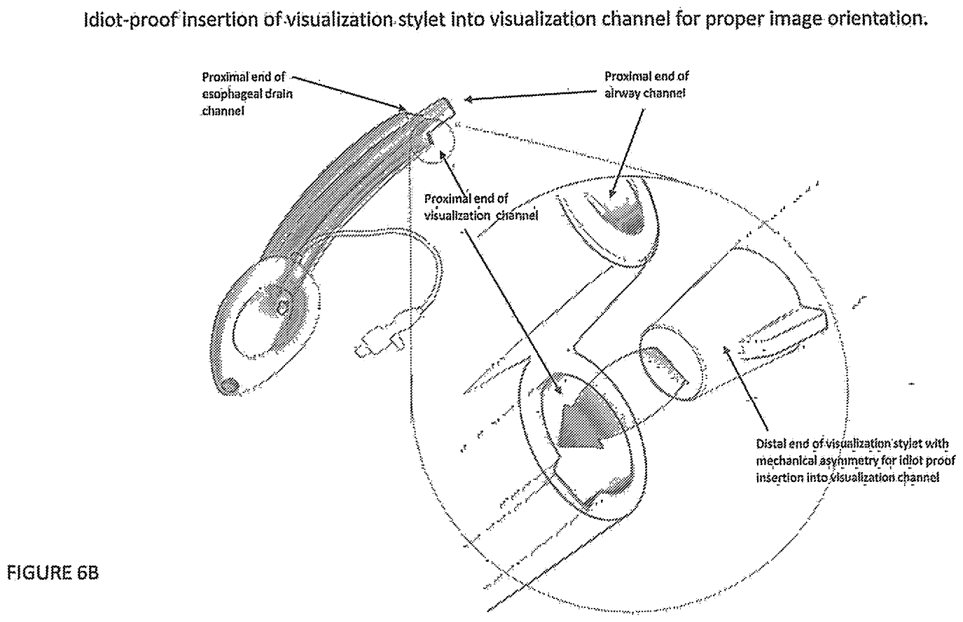

In another embodiment of this invention, once the visualization stylet is communicating with a computer or smart device such an iPhone, iPad, smart watch, or equivalent android hardware (especially wirelessly), the video signal can also be transmitted to a central monitoring station (wirelessly or via wire) from said smart device. Wireless transmission to a central monitoring station can be from either the wireless access point or from the individual station iPad or wireless tablet the trainee is using to visualize ventilation. Such central monitor station can be a computer or another iPad for example which is equipped with proper software so that it can monitor live video signals from multiple visualization stylets (or multiple procedures). In this embodiment, a senior anesthesiologist or a trainer could be simultaneously monitoring multiple LMA insertions or intubations performed by junior staff at the same time and can either intervene when needed or communicate and assist them remotely via texting or audio back to the individual that was identified needing help (see FIG. 6; multiple patients intubated and central monitoring station controlled by supervising physician).

Methods of Correcting the Horizon Viewing:

The visualization stylet and visualization channel of the LMA can be designed with proper asymmetries that ensure that the visualization stylet is inserted with a proper orientation so that the horizon of the viewing on the display is always preserved for the physician (see FIG. 6B; stylet with asymmetric entering the visualization channel that has corresponding mating female tab to ensure idiot proof insertion of the visualization stylet)

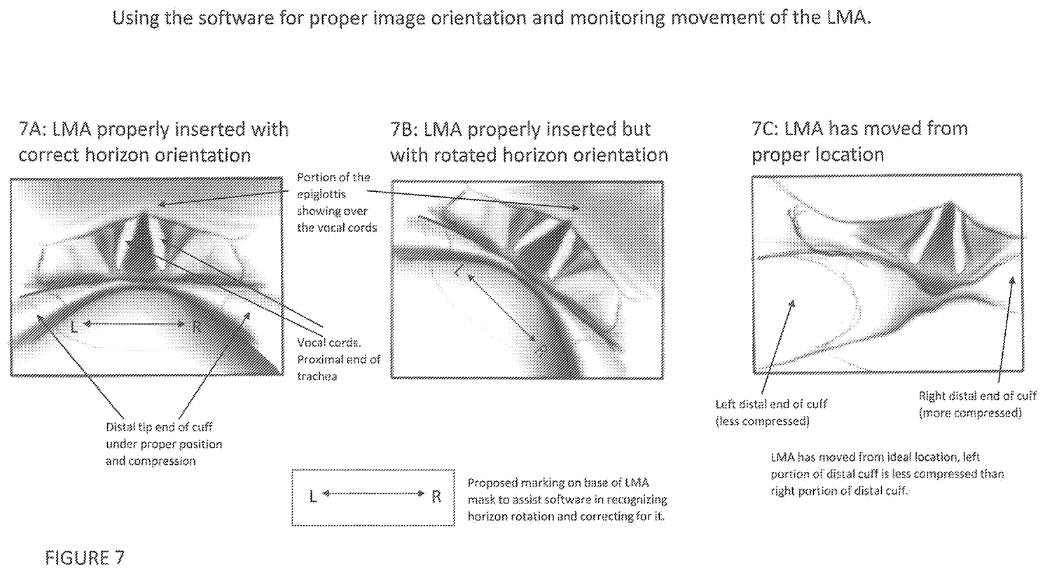

In another embodiment, the visualization stylet and the visualization channel of the LMA may not have any asymmetries. In this case the actual LMA structure itself can aid as a marker to have the software self-orient the image in its proper orientation. For example the visualization channel can have marks that point to the proper orientation. As the symmetric cylindrical stylet is pushed into the visualization channel of the LMA, the software will pick up such markings that will be recognized by image processing algorithms. Then the software can accommodate for the proper rotation of the viewing to ensure that the horizon is preserve so as not to confuse the physician. Similarly, when the visualization stylet is pushed at the end of its channel (the proper location for monitoring LMA placement or ETT passage into the trachea), a portion of the LMA will still be visible along with the patient's anatomy. FIG. 7A shows the proper orientation of the horizon. Thus, the software by looking at the orientation of the LMA tip or markings on the LMA mask (made in locations on the mask or cuff that are clearly still viewable by the visualization stylet, see FIG. 7) can correct automatically for the proper horizon rotation of the scene.

Such horizon correction need not be performed only with the aid of markings inside the visualization channel of the LMA. Rather when the visualization stylet is pushed into the distal end of the visualization channel of the LMA, the image processing software can recognize anatomical structures that come into view. Then through appropriate algorithms, which may include artificial intelligence (AI), the software can recognize what would be the proper orientation of the image by looking at such anatomical structures of the pharynx/larynx and the proximal end of the trachea and appropriately rotating the view into its correct orientation for the physician. See for example FIG. 7B where the symmetrical visualization stylet is inserted and the viewing is rotated. The software can pick this up and correct the horizon by undoing the rotation and making the viewing look like FIG. 7A.

Another aspect of the markings in the LMA, visible via the scope as it is inserted and/or in its inserted position, is that of providing a scale. One or more dimensions of the markings provide reference for the software, and from that scale reference the size of a patient's anatomical features in the region of interest can be calculated. The data from a number of patients can be used to determine proper sizes of the LMA to be used with particular patients.

Continuous Placement Monitoring:

Additionally, since the visualization stylet is in contact with the LMA, should the LMA move after its final placement, the image processing hardware can be calibrated to recognize such movements and present the user with alerts that the LMA has moved from its proper resting place with respect to the anatomy it was originally pushed against. Such alerts can pop up on the display monitor of the tablet, or be wirelessly transmitted to a commercially available tablet, smart phone, or smart watch (iwatch from Apple Inc. for example) or to an external monitor. Such continual optical monitoring of the LMA placement can alleviate concerns that can plague the use of a traditional LMA since LMAs have the tendency to move after final placement and can interrupt the patient's ventilation (see FIG. 7; picture of viewing anatomy and alignment targets etc).

Esophageal Drain Channel:

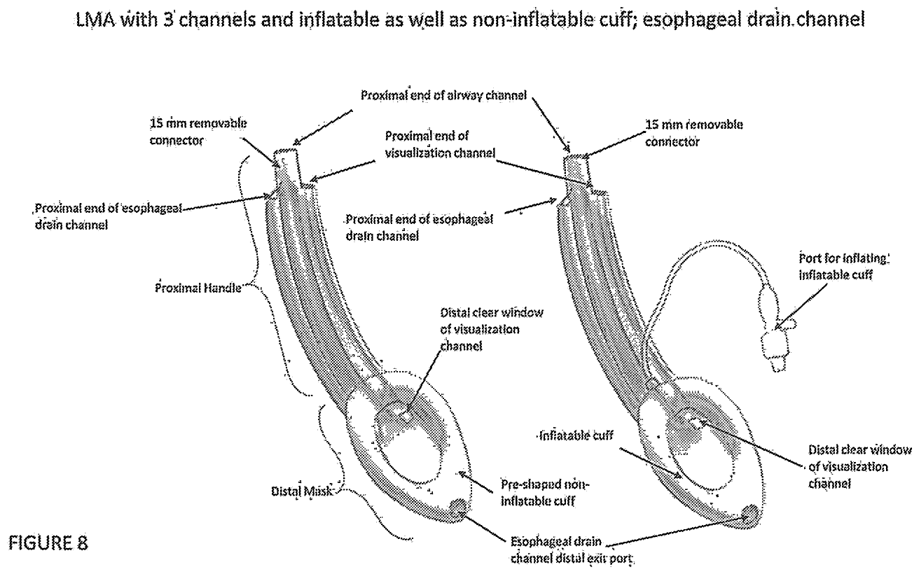

In another embodiment of this invention, a third channel runs also along the length of handle (esophageal drain channel). This channel is communicating with the distal tip end of the LMA mask. Since the distal end of the cuff is designed so that the distal tip of the mask can make a seal with the proximal end of the esophagus, a built-in drain channel (the third channel) can allow expelled gastric content to bypass the pharynx, thus to permit drainage of passively regurgitated gastric fluid away from the airway and serving as a passage for gastric tube. This specific feature is designed to decrease the risk of aspiration. A drain tube can be inserted through this channel all the way into the esophagus for suction. See FIG. 8; esophageal channel, LMA with three channels.

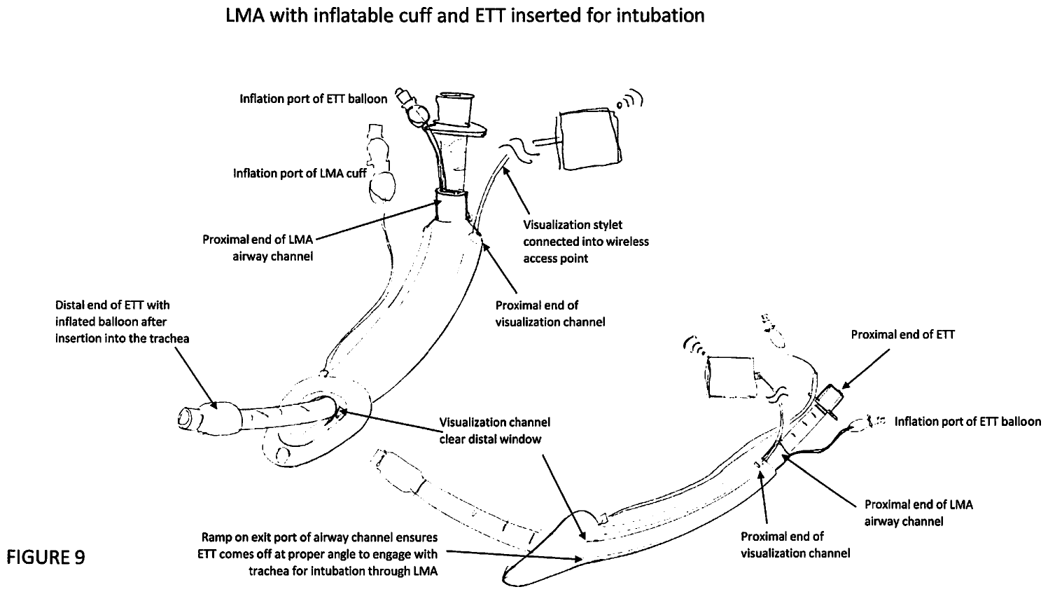

Design of an Intubating LMA (ILMA): In another embodiment of this invention the LMA can also act as an ILMA. The airway channel must be large enough to accept a correspondingly sized ETT. Once the LMA described above is positioned in place, an ETT can be passed through its main airway channel. The 15 mm connector at the proximal end of the airway channel of the LMA may be removable to further aid the insertion of the ETT. The angle of the distal port of the airway channel of this embodiment is designed with a ramp such that when the ETT exits the LMA its distal end is pointing directly into the vocal cords and the proximal end of the trachea (see FIG. 9; ETT coming out of LMA). Once the ETT exits the LMA, it will come into view by the visualization stylet and thus its placement and insertion through the vocal cords and into the proximal end of the trachea can be trivial. In this embodiment, a patient can be intubated easily without the need for a laryngoscope, and in essence the ILMA can act as a laryngoscope replacement.

Removable “Peelable” LMA after ETT Insertion:

In one embodiment of this invention if an ETT is used that is more than twice the total length of the LMA, then once the ETT is in position and its distal balloon inflated to secure it in place, the LMA can be removed by sliding it off the ETT.

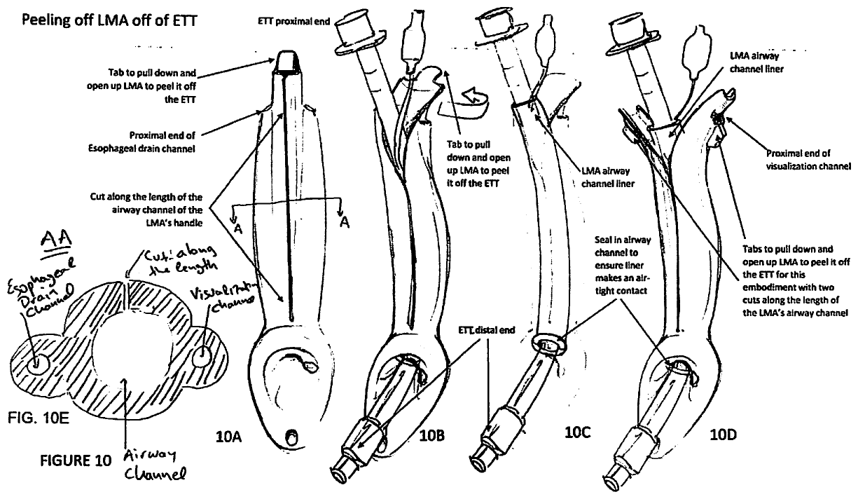

In a preferred embodiment of this invention the LMA is designed with at least one cut along the length of the handle. The cut and the material of the handle portion of the LMA is such that without any radial force on the handle, the handle maintains a seal, or a substantial seal, of the airway channel of the LMA (very slight air leakage is not critical). A releasable adhesive could be applied between the two abutting surfaces for additional sealing. By pulling down on the tab in FIG. 10B, the LMA handle can open (peel open) and expose the installed/secured ETT (see FIG. 10B). This way, once the ETT is in place and secured by inflating its distal balloon inside the trachea, the LMA can be removed by “peeling it off” the ETT and not “sliding it off” the length of the ETT (as in the prior art). Thus, any standard ETT can be used, and removal of the LMA becomes trivial and quickly accomplished. In another embodiment of the “peelable” LMA, the LMA may have more than one cut to further accommodate an even easier removal of the LMA off the ETT (see FIG. 10D). Two tabs can be pulled down from left to right to easier peel open the LMA. FIG. 10E shows one example of the cut LMA airway channel, with an abutting seam.

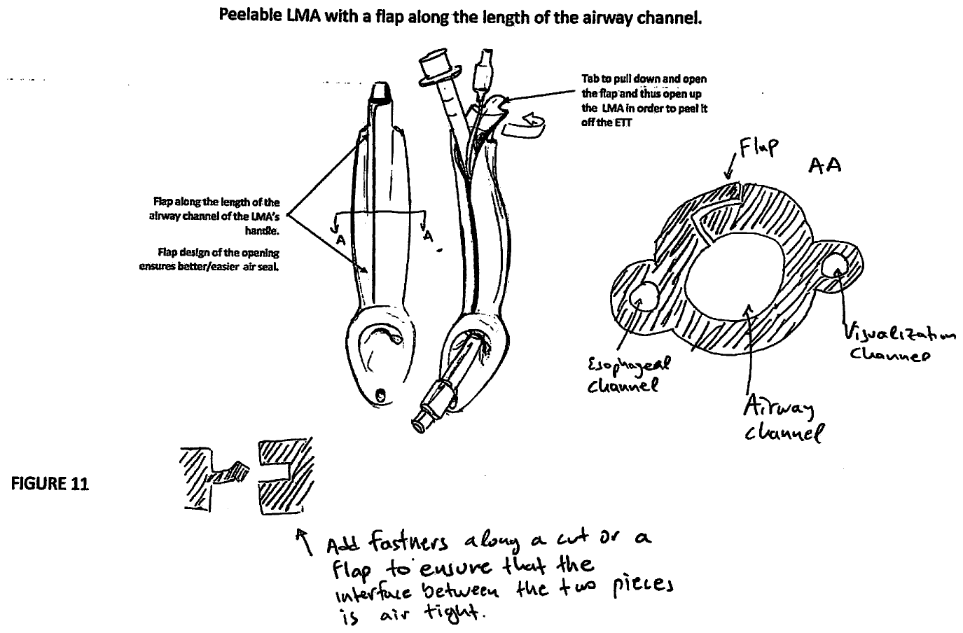

In another embodiment, if the material used for the handle need not be limited to a certain type for which a cut along the length of the airway channel can still maintain airway with no leaks, the cut along its length can be incomplete. There will be a small portion of the thickness of the tubular wall of the LMA handle still intact, through the length of the cut, to prevent air from leaking, yet it is easy to complete the cut by radial pulling on the tabs of FIGS. 10 and 11 to peel the handle apart.

In another embodiment of the “peelable LMA” a thin tube liner can also be inserted in the airway channel of the LMA to ensure the airway is preserved and there are no leaks from the aforementioned cut or cuts on the handle of the LMA along the airway channel that aid in the “peel off” of the LMA from the ETT tube. Such liner must be designed with a thin wall so as not to add significant stiffness to the LMA handle, and it must be communicating with the exit port of the airway channel at the mask so as to not be leaky and to preserve the airway. This can be achieved by designing a proper seal near the distal exit port of the airway channel of the LMA so that the liner can be pushed up against it and wedged into it. It is preferred such liner is also removable (see FIG. 10C). When the LMA is removed by peeling it off the ETT, the sheer lener is easily brought up and off the proximal end of the ETT; it can be bunched together into a compact ring if needed.

In another embodiment of the peelable LMA invention, “peeling” open the LMA from the ETT can be achieved not via a simple cut along the length of the airway channel, but by creating a an overlap along the length of the airway channel of the handle. The overlap can be as shown in FIG. 11 in cross section, and can be opened by pulling the outer flap out. This overlap design can ensure an airway seal, and a releasable adhesive can also be applied, as noted above, for additional sealing integrity. Note also, FIG. 11 shows an alternative wherein a series of fastener tabs insert into corresponding slots to firmly hold the joint together. This could also be a continuous tab or flange inserted in a continuous slot.

Monitoring Changes in the Pressure:

When the LMA is correctly placed in position and the cuff seals around the trachea opening, the pocket in the mask is an area that can monitor the pressure that builds up through the ventilation of the patient.

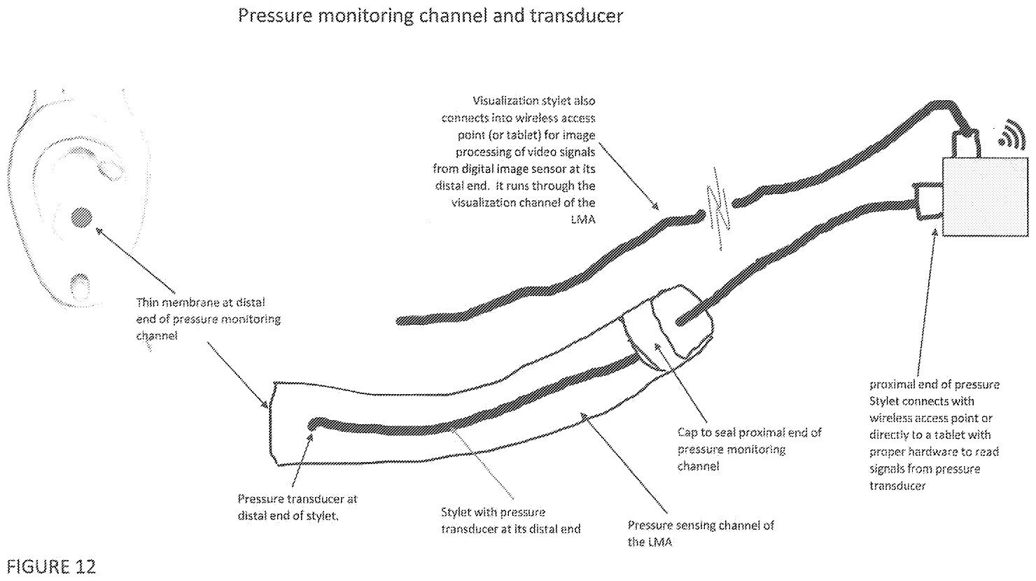

In another embodiment of this invention, the LMA can have a fourth channel (Pressure Monitoring Channel PMC) running along the length of the handle. Such channel is terminated at the distal end of the mask portion of the LMA with a thin polymer membrane “window” (not transparent). It is thin enough so that pressure changes within the mask can modify its shape, but thick enough to maintain a sterile barrier. Another insertable flexible stylet with a pressure transducer at its distal end can be inserted through this fourth channel and the proximal end of the PMC can be locked/sealed at the proximal end of the handle to prevent atmospheric pressure changes from modifying the shape of the distal membrane of the PMC. Thus, only changes in the pressure below the mask of the LMA can affect the shape of such membrane window, and this changes the pressure within this closed chamber. The membrane window of the stylet provides a sterile barrier, so the pressure stylet does not need to be reprocessed after every use. Any type of pressure transducer can be placed on the tip of the pressure stylet. Such transducer can be electrical, electronic, mechanical, optical, or any other pressure transducer than can be made small enough to fit at the tip of the pressure monitoring stylet. The proximal end of the pressure monitoring stylet can be connected to the wireless access point or display tablet that has corresponding hardware to read the signals from the pressure transducer and monitor changes in the pressure of the air pocket between the mask and the tissue sealed by the cuff of the LMA mask. Large changes in the pressure within the mask can indicate that the LMA moved. If certain pressure cannot be maintained, the software can instruct the user that the LMA was not placed correctly and needs to be repositioned. Such signal can prove an invaluable tool in monitoring the performance of the LMA and as a training tool to instruct novices of the proper placement of an LMA (see FIG. 12).

In another embodiment of this invention, there is no membrane window at the distal end of the PMC. The distal end of the insertable stylet with the pressure transducer attached to it can then directly sense changes in the pressure within the mask. In this embodiment the pressure stylet will have to be reprocessed after every use.

In yet another embodiment, the pressure transducer can be permanently embedded within the LMA with its sensing element directly exposed to the inside pocket of the mask of the LMA, i.e. to the space below the LMA mask.

Once the LMA is placed correctly and the patient is ventilated, the imaging sensor at the distal end of the visualization stylet can continuously monitor the tissue and anatomical structures engulfed by the cuff of the LMA. Such tissue includes the opening of the trachea and vocal cords (FIG. 7) as well as mucus and saliva and bubbles on that tissue. In another embodiment of this invention, the live video from the imaging sensor can continually monitor anatomical structures such as the tissue around the trachea, mucus, saliva, bubbles on the tissue, or the size of the opening through the vocal cords. Pressure changes can modify the shape of such features and structures and the software can be trained and calibrated to measure pressure changes within the mask air chamber by monitoring changes on the aforementioned anatomical structures.

The above described preferred embodiments are intended to illustrate the principles of the invention, but not to limit its scope. Other embodiments and variations to these preferred embodiments will be apparent to those skilled in the art and may be made without departing from the spirit and scope of the invention as defined in the following claims.

I claim:

1. A laryngeal mask airway device to facilitate pulmonary breathing of a patient and to receive and correctly place an endotracheal tube inserted through the device, comprising:
an airway tube having proximal and distal ends,
a mask attached to the distal end of the airway tube, the mask including a peripheral cuff configured to form a seal around the periphery of the patient's laryngeal inlet,
an endoscope channel formed separately from and alongside the airway tube, configured to receive an endoscope capable of illuminating and viewing the laryngeal anatomy when fully inserted in the channel, and
the endoscope channel being closed at a distal end, with an essentially transparent viewing window to allow the endoscope to illuminate and view tissue distal of the window, such that the endoscope channel remains isolated from the patient's internal tissues so that the endoscope can be reused without sterilization or reprocessing.

2. The laryngeal mask airway device of claim 1, wherein the window of the endoscope channel is positioned so as to enable monitoring of the insertion of an endotracheal tube through said airway tube and into the trachea as the endotracheal tube exits distally from the airway tube.

3. The laryngeal mask airway device of claim 1, wherein the window of the endoscope channel includes means for preventing back reflection of light from the scope.

4. The laryngeal mask airway device of claim 3, wherein the means for preventing back reflection includes the window of the endoscope channel having a scope-receiving portion extending distally a different distance than an illumination portion of the window, to receive a visualization stylet having a mating distal end with a camera/lens essentially extending distally a different distance than the illumination source.

5. The laryngeal mask airway device of claim 1, wherein the endoscope channel includes a mechanical asymmetry to assure proper insertion of the visualization stylet and rotational orientation of images from the visualization stylet.

6. The laryngeal mask airway device of claim 1, further including an additional channel as a pressure channel for monitoring pressure in the laryngeal region of the patient, distal of the mask and cuff, the pressure channel configured to receive a pressure sensing device.

7. The laryngeal mask of claim 6, wherein the pressure channel is closed-ended with a movable membrane closing the distal end of the pressure channel, so that pressure changes in the laryngeal region are communicated by the flexible membrane to the interior of the pressure channel so that pressure changes can be determined via a sensor in the pressure channel.

8. The laryngeal mask airway device of claim 1, further including an esophageal drainage channel formed separate from and alongside the airway tube.

9. The laryngeal mask airway device of claim 1, wherein the airway tube has an elongated seam, essentially through the length of the airway tube, configured to allow separation of the airway tube along the seam after placement of an endotracheal tube through the airway tube, by pulling the airway tube apart at the seam.

10. The laryngeal mask airway device of claim 1, in combination with a visualization stylet, the visualization stylet having wireless means for connection to a monitor for display of images from the visualization stylet when fully inserted into the endoscope channel.

11. The laryngeal mask airway device of claim 1, wherein the mask includes markings in position to be imaged by a visualization stylet inserted through the endoscope channel, and the visualization stylet being connected to a programmed computer to which a monitor is connected for displaying of images, and the programmed computer including means for determining rotational orientation of the visualization stylet via the markings, and correction means for correcting orientation of an image displayed on the monitor to a normal orientation, regardless of rotational orientation of the visualization stylet.

12. The laryngeal mask airway device of claim 1, wherein the mask includes markings in position to be imaged by a visualization stylet inserted through the endoscope channel, and the visualization stylet being connected to a programmed computer to which a monitor is connected for displaying of images, and the programmed computer including means for reading images of the markings to provide a known dimensional reference or scale, and for imaging a patient's anatomical features in the laryngeal and tracheal region to assess size of the patient's anatomical features.

13. A method for accurate emplacement of an endotracheal tube into a laryngeal region of a patient, comprising:
emplacing into the patient a laryngeal mask airway device having an airway tube with proximal and distal ends, a mask attached to the distal end of the airway tube, and a peripheral cuff forming a part of the mask, and assuring that the peripheral cuff is correctly emplaced to form a seal around the patient's laryngeal inlet,
the laryngeal mask airway device including an endoscope channel separate from the airway tube and extending to the distal end of the laryngeal mask airway device,
placing a visualization stylet through the endoscope channel to locate a distal end of the stylet at the cuff in position to view laryngeal anatomy and a distal end of the airway tube, the visualization stylet being connected to a computer with imaging means and to a video display monitor,
extending the endotracheal tube down through the airway tube until a distal end of the endotracheal tube extends out the distal end of the airway tube and through the cuff, and
while extending the endotracheal tube, using the visualization stylet and observing on the monitor progress of the endotracheal tube as it is extended out of the cuff and toward the trachea of the patient, and adjusting the endotracheal tube as needed based on images displayed on the viewing monitor, to ensure accurate placement of the endotracheal tube.

14. A method for training medical personnel in emplacement of endotracheal tubes in patients, comprising:
providing a series of laryngeal mask airway devices, each including an airway tube with proximal and distal ends, endoscope channel separate from the airway tube and extending to the distal end of the laryngeal mask airway device, so that a laryngeal and tracheal region of a patient can be viewed when a scope is inserted,
providing a visualization stylet for use with each of the laryngeal mask airway devices,
providing an endotracheal tube for use with each of the laryngeal mask airway devices,
furnishing each of a series of trainee medical personnel with a laryngeal mask airway device, a visualization stylet and an endotracheal tube, and while each of the trainees works to place the laryngeal mask airway device and endotracheal tube in a patient or simulated patient, a supervisor's monitoring the actions of all trainees simultaneously using a computer monitor connected wirelessly to each of the visualization stylets so as to have presented to the supervisor images from each of the visualization stylets as the laryngeal mask airway device is emplaced in a patient and as the endotracheal tube is inserted through the airway tube and toward the trachea of the patient or simulated patient, and
via two-way communication between a supervisor and each of the trainees, the supervisor's advising the trainees on intubation and correcting actions taken by the trainees.
